(19) United States
(12) Patent Application Publication
(10) Pub. No.: US 2024/0065728 A1
(43) Pub. Date: Feb. 29, 2024

(54) EXPANDABLE CANNULA ASSEMBLY

(71) Applicant: LSI Solutions, Inc., Victor, NY (US)

(72) Inventor: Jude S. Sauer, Pittsford, NY (US)

(21) Appl. No.: 18/236,787

(22) Filed: Aug. 22, 2023

Related U.S. Application Data
(60) Provisional application No. 63/400,111, filed on Aug. 23, 2022.

(51) Int. Cl. A61B 17/34

(52) U.S. Cl. CPC A61B 17/3439; A61M 25/0023

(57) ABSTRACT

An expandable cannula assembly includes a first cannula subassembly comprising an upper body member having a first upper body mating feature and an upper lateral panel coupled to the first upper body member which includes a first upper lateral panel mating feature. The assembly further includes a second cannula subassembly comprising a lower body member having a first lower body mating feature and a lower lateral panel coupled to the lower body member which includes a first lower lateral panel mating feature. The first lower lateral panel mating feature operatively engages the first upper body mating feature, and the first upper lateral panel mating feature operatively engages the first lower body mating feature such that the first cannula subassembly and the second cannula assembly are displaceable between a first position having a first cross-sectional shape and a second position having a second cross-sectional shape.

### CROSS-REFERENCE TO RELATED APPLICATIONS

This application claims the benefit of U.S. Provisional Patent Application No. 63/400,111, filed Aug. 23, 2022, which is incorporated by reference herein in its entirety.

### FIELD OF THE INVENTION

The claimed invention relates to surgical devices, and more specifically to a cannula assembly.

### BACKGROUND OF THE INVENTION

Cannulas are typically used during minimally-invasive surgical procedures to insert one or more instruments through an incision through a patient to a treatment area of the patient. The cannula typically guides the one or more instruments to the treatment area and allows the one or more instrument to pass through the incision without additional trauma to the incision area. Because the instruments used in the procedure may sometimes have large distal ends, the diameter or cross-sectional shape of the cannula must be large enough to accommodate the largest of such instruments. However, such a relatively large cross-sectional shape of the cannula requires a large incision in the patient, and large incision is maintained as long as the cannula passes through the incision of the patient. The resulting relatively large incision size needed to accommodate the cannula results in increased recovery time for a patient, but can also lead to pain, an increased risk of infection, and other disadvantageous outcomes for the patient during recovery. Accordingly, there is a need for a cannula that can accommodate large instrument during a procedure but minimizes the trauma to the incision area.

### BRIEF DESCRIPTION OF THE DRAWINGS

FIGS. 1A to 2D are various views of an embodiment of an expandable cannula assembly in a first position;

FIG. 3A is a cross-sectional view of the embodiment of the expandable cannula assembly of FIG. 1B taken along section line 3A-3A;

FIG. 3B is a perspective view of the cross-sectional view of FIG. 3A;

FIG. 3C is a cross-sectional view of the embodiment of the expandable cannula assembly of FIG. 1B taken along section line 3C-3C;

FIG. 3D is a perspective view of the cross-sectional view of FIG. 3C;

FIGS. 4A to 4C are various views of an embodiment of a first cannula subassembly of the expandable cannula assembly;

FIGS. 5A and 5B are various views of the embodiment of the first cannula subassembly of the expandable cannula assembly coupled with the lower body member of the second cannula subassembly, with the lower lateral panel of the second cannula subassembly omitted for clarity;

FIGS. 6A to 6C are various views of an embodiment of a second cannula subassembly of the expandable cannula assembly;

FIG. 6D is a side view of a second protrusion of a lower lateral panel of the second cannula subassembly;

FIG. 7 is a perspective view of the embodiment of the second cannula subassembly of the expandable cannula assembly coupled with the upper body member of the first cannula subassembly, with the upper lateral panel of the first cannula subassembly omitted for clarity;

FIG. 8A is a front view of the embodiment of the first cannula subassembly and the second cannula subassembly of the expandable cannula assembly in a second position;

FIG. 8B is a front view of the embodiment of the first cannula subassembly and the second cannula subassembly of the expandable cannula assembly in a third position between the second position and the first position;

FIG. 8C is a perspective view of the embodiment of the first cannula subassembly and the second cannula subassembly of the expandable cannula assembly in the first position;

FIGS. 9A to 9B are top and side views, respectively, of an embodiment of an upper body member of the first cannula subassembly;

FIG. 9C is a cross-sectional view of the embodiment of the upper body member of FIG. 9B taken along section line 9C-9C;

FIG. 9D to 9F are perspective views of the embodiment of the upper body member of FIG. 9B;

FIGS. 10A to 10B are top and side views, respectively, of an embodiment of a lower body member of the second cannula subassembly;

FIG. 10C is a cross-sectional view of the embodiment of the lower body member of FIG. 10A taken along section line 10C-10C;

FIG. 10D is a perspective view of the cross-section of the lower body member of FIG. 10A taken along section line 10D-10D; and

FIG. 11 is a side view of a distal end of a medical instrument that is to be inserted into an end of an embodiment of the expandable cannula assembly in the first position, with the expandable cannula assembly maintained in the first position prior to the insertion of the distal end of the instrument for illustrative reasons.

### DETAILED DESCRIPTION OF THE INVENTION

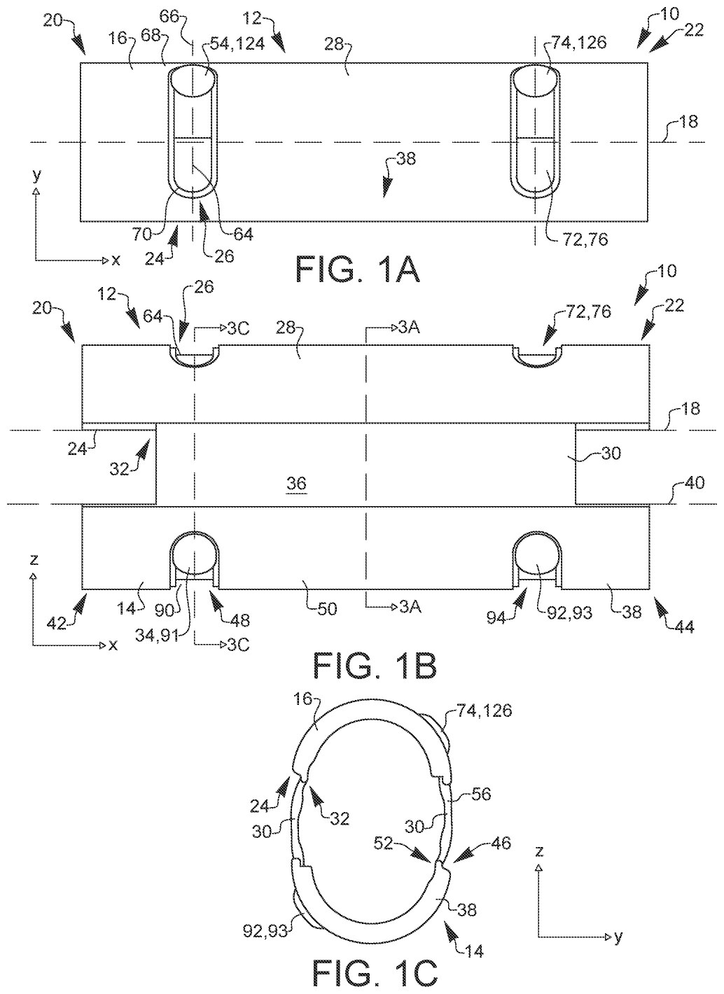

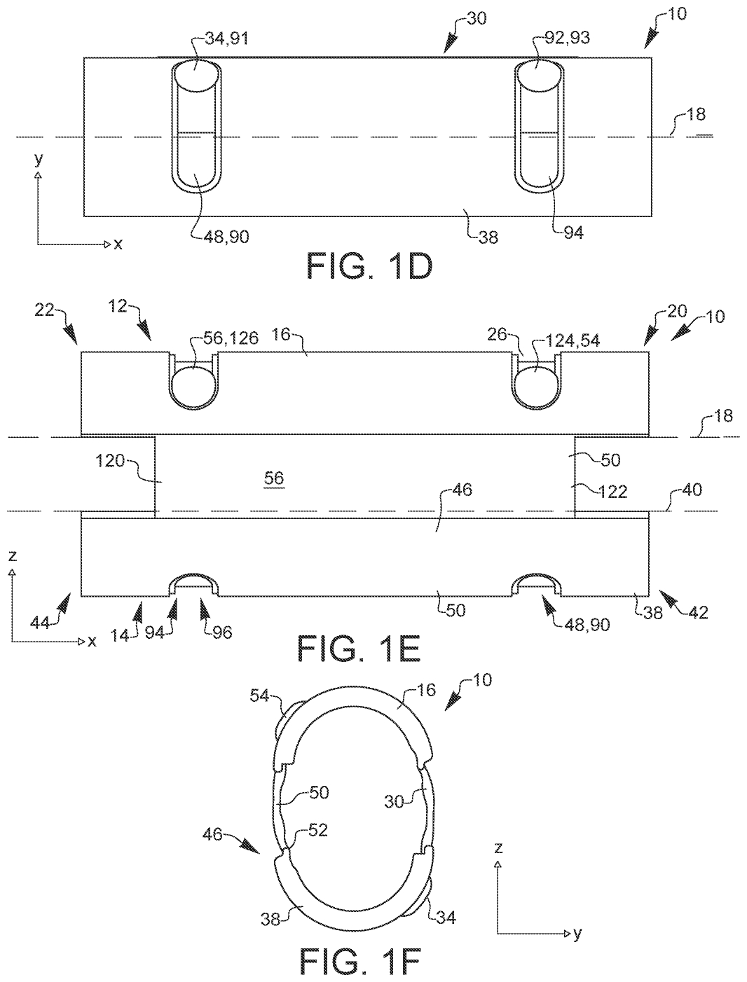

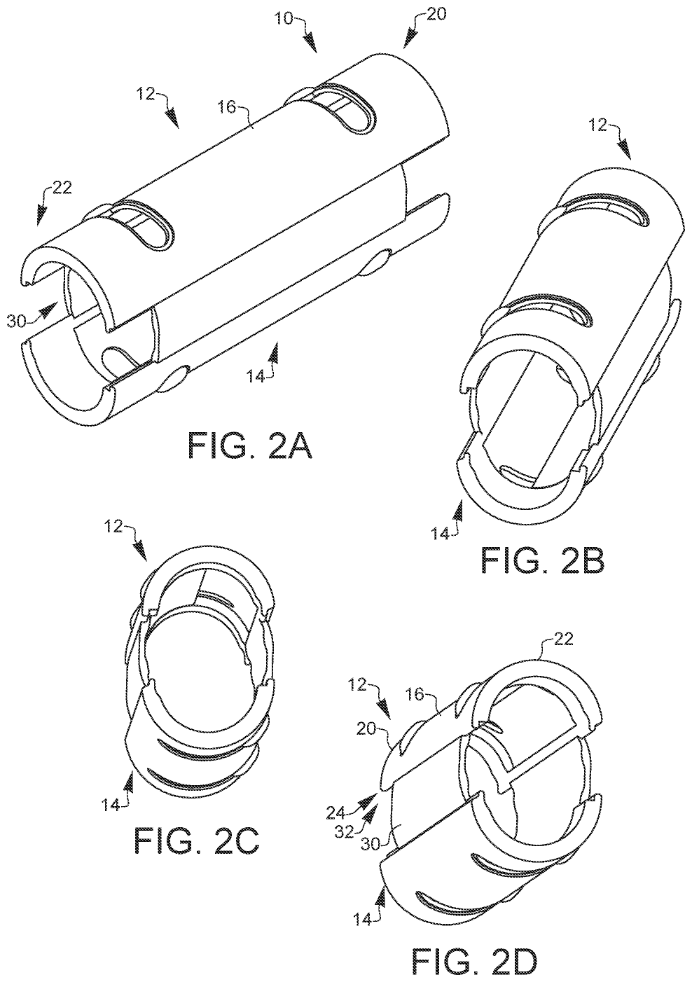

As illustrated in FIGS. 1A to 2D, an embodiment of an expandable cannula assembly 10 includes a first cannula subassembly 12 and a second cannula subassembly 14 that is movably coupled to the first cannula subassembly 12. With reference to FIG. 1B, the first cannula subassembly 12 includes an upper body member 16 that extends along an upper body axis 18 from a first end 20 to a second end 22, the upper body member 16 having a first body edge portion 24 that extends parallel to and offset from the upper body axis 18, and a first upper body mating feature 26 is disposed along a first portion of a surface 28 of the upper body member 16. The first cannula subassembly 12 also includes an upper lateral panel 30 having a first panel edge 32 (see FIG. 1C) that is coupled to the upper body member 16 at or adjacent to the first body edge portion 24 of the upper body member 16, and a first upper lateral panel mating feature 34 is disposed on a first portion of a surface 36 of the upper lateral panel 30.

Still referring to FIG. 1B, the second cannula subassembly 14 includes a lower body member 38 that extends along a lower body axis 40 from a first end 42 to a second end 44. With reference to FIG. 1E, the lower body member 38 having a first body edge portion 46 that extends parallel to and offset from the lower body axis 40, wherein a first lower body mating feature 48 is disposed along a first portion of a surface 50 of the lower body member 38. The second cannula subassembly 14 also includes a lower lateral panel 50 having a first panel edge 52 that is coupled to the lower body member 38 at or adjacent to the first body edge portion 46 of the lower body member 38 (see FIG. 1F), and a first lower lateral panel mating feature 54 is disposed on a first portion of a surface 56 of the lower lateral panel 50.

As illustrated in FIG. 1E, the first lower lateral panel mating feature 54 operatively engages the first upper body mating feature 26 such that the first lower lateral panel mating feature 54 is displaceable relative to the first upper body mating feature 26. With reference to FIG. 1B, the first upper lateral panel mating feature 34 operatively engages the first lower body mating feature 48 such that the first upper lateral panel mating feature 34 is displaceable relative to the first lower body mating feature 48. So configured, the first cannula subassembly 12 and the second cannula assembly 14 are displaceable between a first position (or a deployed position, illustrated in FIGS. 1A to 2D) and a second position (or an undeployed position, illustrated in FIG. 8A). In the first position, the first cannula subassembly 12 and the second cannula assembly 14 have a first cross-sectional assembly shape and in the second position, the first cannula subassembly 12 and the second cannula assembly 14 have a second cross-sectional assembly shape that is different than the first cross-sectional shape.

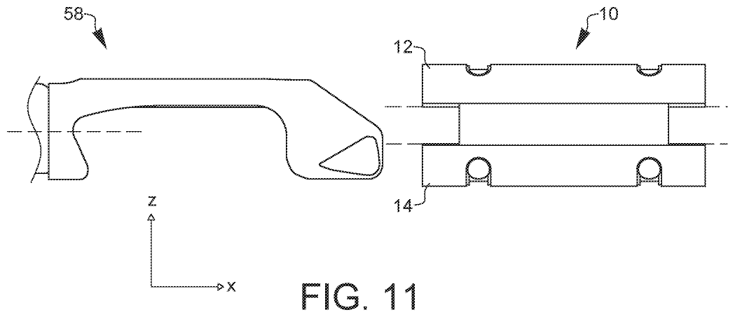

In use during a procedure, the expandable cannula assembly 10 may be inserted into an incision of a patient in the second position to minimize trauma to the incision at the area of insertion. However, if a relatively large instrument (such as the instrument 58 illustrated in FIG. 11) must be passed through the expandable cannula assembly 10, the first cannula subassembly 12 and the second cannula assembly 14 may transition from the second position into the “deployed position,” or the first position, to accommodate the large instrument 58, and the transition from the second position to the first position will be discussed in more detail in a following section. When the instrument 58 is removed from the expandable cannula assembly 10, the first cannula subassembly 12 and the second cannula assembly 14 may quickly transition back to the second position, thereby minimizing the time in which the expandable cannula assembly 10 is in the more traumatic first position.

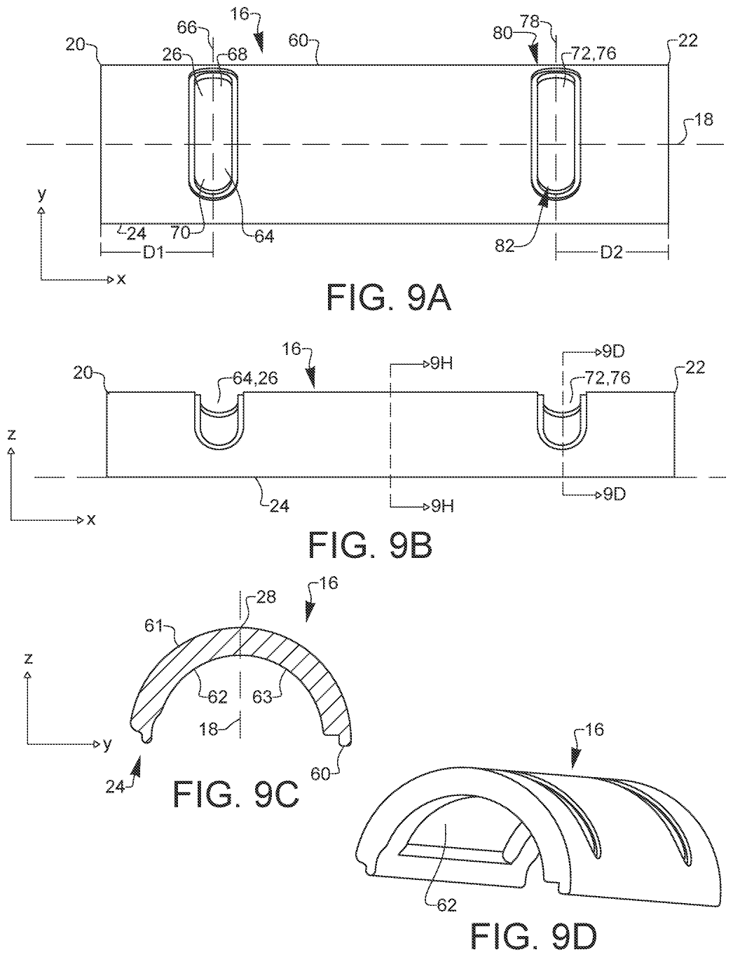

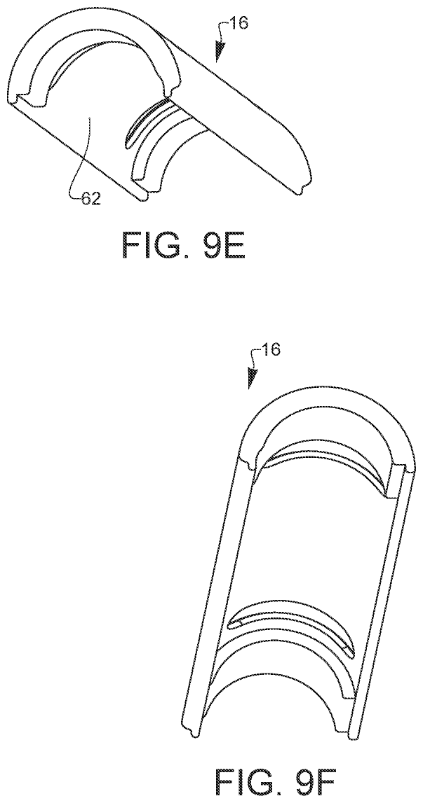

Turning to the first cannula subassembly 12 of the expandable cannula assembly 10 in more detail, FIG. 9A illustrates the upper body member 16 that extends along the upper body axis 18 from the first end 20 to the second end 22. The upper body axis 18 extends parallel to or along the X-axis of the reference coordinate system of FIGS. 9A to 9C. The upper body member 16 may have any suitable cross-sectional shape or combination of shapes. In particular, when viewed in a cross-section that is normal to the X-axis, such as a cross-sectional plane that is parallel to the Y-Z plane of the reference coordinate system of FIGS. 9A to 9C, the upper body member 16 may have an arcuate, semi-circular, or partially-circular cross-sectional shape. That is, in cross section, the upper body member 16 may extend from the first body edge portion 24 to a second body edge portion 60 along a path or axis that may be a segment of a circle, such as one half of a circle, as illustrated in FIG. 9C. The cross-sectional shape may be further defined by a first edge 61 defined by the surface 28 (for example, the exterior surface) and second edge 63 defined by an interior surface 62. The cross-sectional shape may be bisected by a plane that contains the upper body axis 18 and that is parallel to the X-Z plane of the reference coordinate system of FIGS. 9A to 9C. The cross-sectional shape may be uniform or substantially uniform along the upper body axis 18 from the first end 20 of the upper body member 16 to the second end 22 of the upper body member 16.

As illustrated in FIG. 9A, the upper body member 16 may include the first upper body mating feature 26 that may be disposed along the first portion of the surface 28 of the upper body member 16. The first upper body mating feature 26 may be any feature that may engage the first lower lateral panel mating feature 54 such that the first lower lateral panel mating feature 54 is displaceable relative to the first upper body mating feature 26. For example, the first upper body mating feature 26 may be a first slot 64 is disposed along the first portion of the surface 28 of the upper body member 16, and the first slot 64 of the upper body member 16 may extend along a first slot axis 66 from a first end 68 to a second end 70. The first slot axis 66 may extend parallel to the Y-axis of the reference coordinate system of FIGS. 9A to 9C when viewed along the Z-axis. However, the first slot axis 66 may conform in shape to the cross-sectional shape of the upper body member 16 when viewed in a cross-section that is normal to the X-axis, such as a cross-sectional plane that is parallel to the Y-Z plane of the reference coordinate system of FIGS. 9A to 9C. The first slot 64 may be defined by first and second lateral edges that are parallel, and the first and second ends 68, 70 may be defined by a first and second semi-circular edge, respectively. The first slot 64 may be disposed any distance from the first end 20 of the upper body member 16. In particular, the first slot axis 66 may be disposed a first distance D1 from the first end 20 of the upper body member 16.

Still referring to FIG. 9A, the upper body member 16 may include a second upper body mating feature 72 that may be disposed along a second portion of the surface 28 of the upper body member 16. The second upper body mating feature 72 may be any feature that may engage a second lower lateral panel mating feature 74 such that the second lower lateral panel mating feature 74 is displaceable relative to the second upper body mating feature 72. In some embodiments, the second upper body mating feature 72 may be identical to the first upper body mating feature 26. Specifically, the second upper body mating feature 72 may be a second slot 76 disposed along the second portion of the surface 28 of the upper body member 16, and the second slot 76 of the upper body member 16 may extend along a first slot axis 78 from a first end 80 to a second end 82. The second slot axis 78 may extend parallel to the Y-axis of the reference coordinate system of FIGS. 9A to 9C (and to the first slot axis 66) when viewed along the Z-axis. However, identical to the first slot axis 66, the second slot axis 78 may conform in shape to the cross-sectional shape of the upper body member 16 when viewed in a cross-section that is normal to the X-axis, such as a cross-sectional plane that is parallel to the Y-Z plane of the reference coordinate system of FIGS. 9A to 9C. The second slot axis 78 may be defined by first and second lateral edges that are parallel, and the first and second ends 80, 82 may be defined by a first and second semi-circular edge, respectively. The second slot axis 78 may be disposed any distance from the second end 22 of the upper body member 16. In particular, the second slot axis 78 may be disposed a second distance D2 from the second end 22 of the upper body member 16, and the second distance D2 may be equal to the first distance D1 between the first slot axis 66 and the first end 20 of the upper body member 16.

As illustrated in FIG. 1B, the first cannula subassembly 12 also includes the upper lateral panel 30 having a first panel edge 32 (see FIG. 1C) disposed at a first end of the upper lateral panel 30, and the first panel edge 32 (or any portion of the first end of the upper lateral panel 30) may be coupled to the upper body member 16 at or adjacent to the first body edge portion 24 of the upper body member 16. The first panel edge 32 of the upper lateral panel 30 may be coupled to the upper body member 16 in any suitable manner. For example, the first panel edge 32 of the upper lateral panel 30 may be fixedly coupled to the upper body member 16 by an adhesive, ultrasonic welding, or by mechanical coupling, such as snap-fit or friction fit coupling. In other embodiments, the first panel edge 32 of the upper lateral panel 30 may be coupled to the upper body member 16 by a hinge or flexible coupling. In still further embodiments, the first panel edge 32 of the upper lateral panel 30 may be integrally formed with the upper body member 16 at or adjacent to the first body edge portion 24 of the upper body member 16.

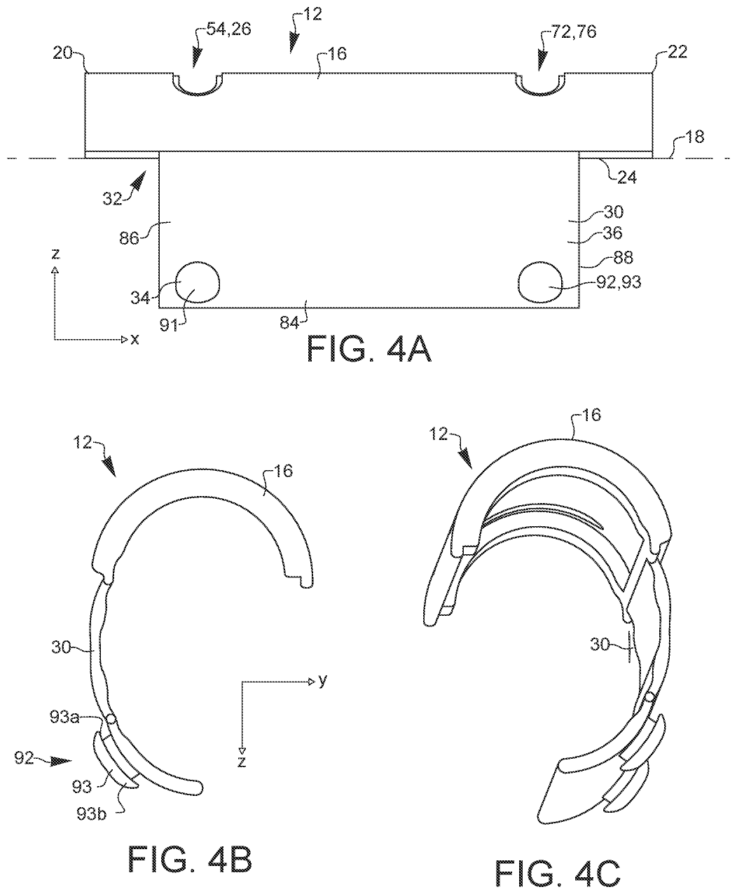

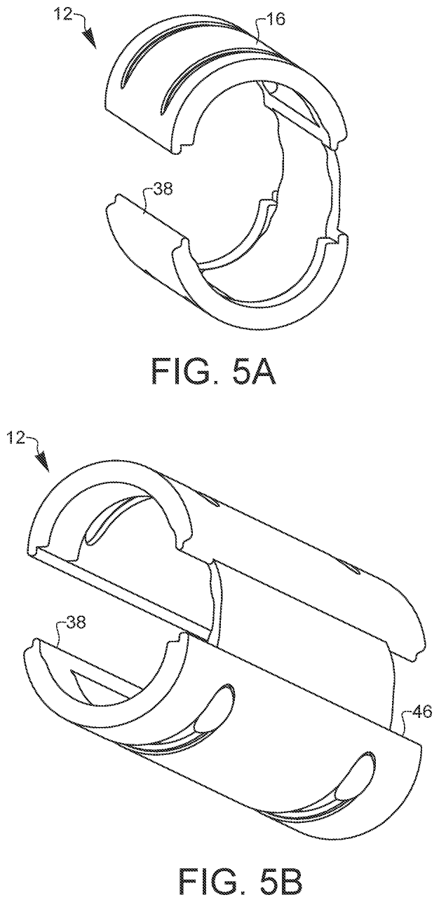

The upper lateral panel 30 may have any suitable shape or combination of shapes. In the embodiment of FIG. 4A, the upper lateral panel 30 may be defined by the first panel edge 32 and a second panel edge 84 that may be parallel (or substantially parallel) to the first panel edge 32 and/or the X-axis of the reference coordinate system of FIGS. 1B and 4A. The second panel edge 84 may define a second end of the upper lateral panel 30. The upper lateral panel 30 may be further defined by a first lateral edge 86 and a second lateral edge 88, where the first lateral edge 86 extends from a first end of the first panel edge 32 to a first end of the second panel edge 84 and the second lateral edge 88 extends from a second end of the first panel edge 32 to a second end of the second panel edge 84. Each of the first lateral edge 86 and the second lateral edge 88 may extend normal to the X-axis of the reference coordinate system of FIGS. 1B and 4A (i.e., along or parallel to the Z-axis). Accordingly, the upper lateral panel 30 may have a square or rectangular shape (or a substantially square or rectangular shape).

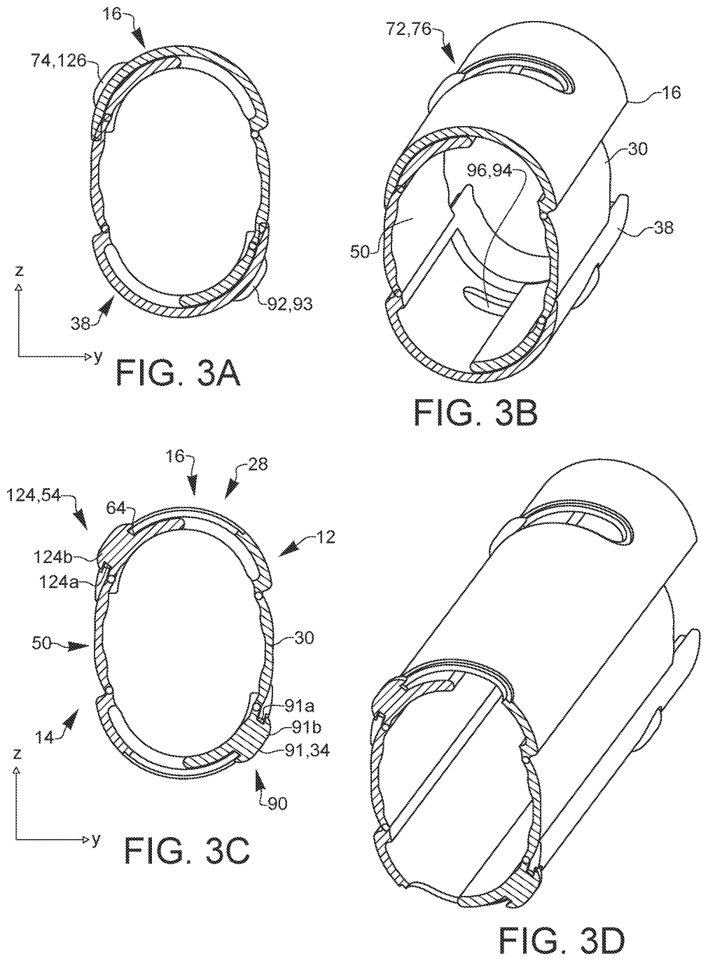

The upper lateral panel 30 may also have the first upper lateral panel mating feature 34 that is disposed on the first portion of the surface 36 (i.e., the outside surface) of the upper lateral panel 30. The first upper lateral panel mating feature 34 may be any feature that may engage the first lower body mating feature 48 of the lower body member 38 (illustrated in FIG. 1B) such that the first upper lateral panel mating feature 34 is displaceable relative to the first lower body mating feature 48. For example, the first upper lateral panel mating feature 34 may be a first protrusion 91 that may be adapted to be received in a first slot 90 disposed along the surface 50 of the lower body member 38, and the first slot 90 will be described in more detail in a following section. As illustrated in the cross-sectional view of FIG. 3C, the first protrusion 91 may have a base portion 91a that may be cylindrical and may have a diameter that is slightly smaller than the distance between the lateral edges of the first slot 90 such that the first protrusion 91 may translate longitudinally within the first slot 90. The first protrusion 91 may also have a top portion 91b coupled to the base portion 91a, and the top portion 91b may have the shape of a disk and may have a diameter that is larger than the distance between the lateral edges of the first slot 90 such that the first protrusion 91 is retained in the first slot 90 as the first protrusion 91 translates longitudinally within the first slot 90. The first protrusion 91 may be disposed adjacent to the second panel edge 84 (or the second end) of the upper lateral panel 30 and may be positioned to align with the first lower body mating feature 48 of the lower body member 38.

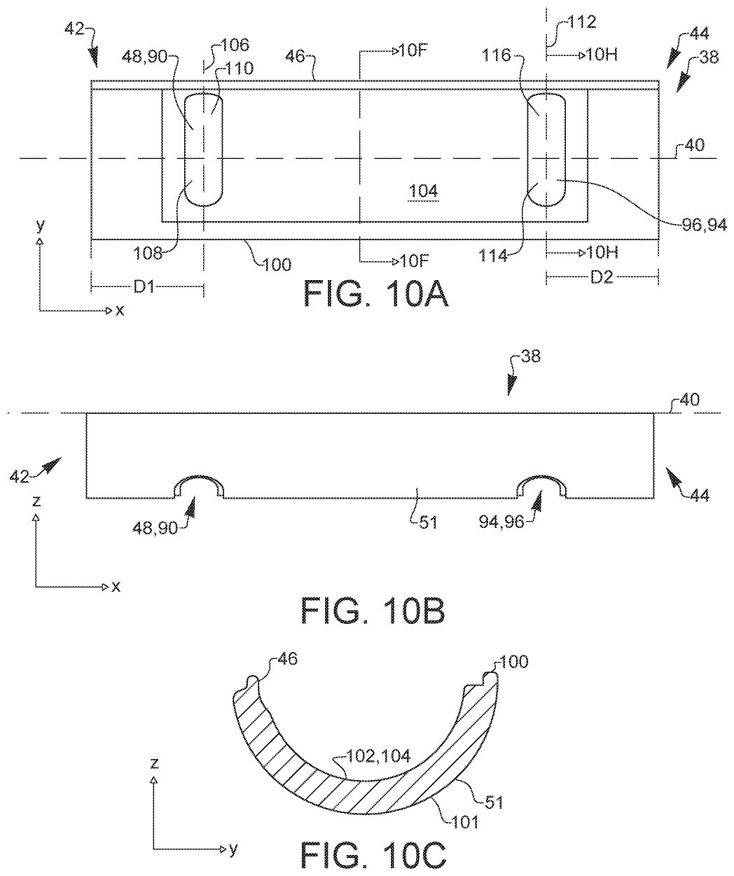

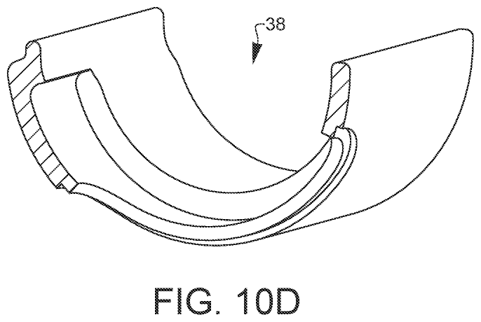

The upper lateral panel 30 may also have a second upper lateral panel mating feature 92 that is disposed on a second portion of the surface 36 (i.e., the outside surface) of the upper lateral panel 30. The second upper lateral panel mating feature 92 may be any feature that may engage a second lower body mating feature 96 of the lower body member 38 (illustrated in FIG. 1B) such that the second upper lateral panel mating feature 92 is displaceable relative to the second lower body mating feature 96. In some embodiments, the second upper lateral panel mating feature 92 may be identical to the first upper lateral panel mating feature 34. For example, the second upper lateral panel mating feature 92 may be a second protrusion 93 that that may be adapted to be received in a second slot 94 (see FIG. 1B) disposed along a second portion of the surface 50 of the lower body member 38, and the second slot 94 may be identical to the first slot 90 and will be described in more detail in a following section. As illustrated in FIG. 4B, the second protrusion 93 may be identical to the first protrusion 91 and may have a base portion 93a that may be cylindrical and may have a diameter that is slightly smaller than the distance between the lateral edges of the second slot 94 (see FIG. 10A) such that the second protrusion 93 may translate longitudinally within the second slot 94. The second protrusion 93 may also have a top portion 93b coupled to the base portion 93a, and the top portion 93b may have the shape of a disk and may have a diameter that is larger than the distance between the lateral edges of the second slot 94 (see FIG. 10A) such that the second protrusion 93 is retained in the second slot 94 as the second protrusion 93 translates longitudinally within the second slot 94. As illustrated in FIG. 4A, the second protrusion 93 may be disposed adjacent to the second panel edge 84 (or the second end) of the upper lateral panel 30 and may be positioned to align with the second lower body mating feature 96 of the lower body member 38.

The upper lateral panel 30 may be made from any suitable material, such as a flexible material. In some embodiments, the upper lateral panel 30 is configured to deform along an axis that is parallel to the upper body axis 18. In some embodiments, the upper lateral panel 30 may be made from a resilient material that may deform in a direction normal to the parallel to the upper body axis 18.

Turning to the second cannula subassembly 14 of the expandable cannula assembly 10 in more detail, FIG. 10A illustrates the lower body member 38 that may be identical (or similar) to the upper body member 16. In particular, the lower body member 38 may extend along the lower body axis 40 from the first end 42 to the second end 44. The lower body axis 40 extends parallel to or along the X-axis of the reference coordinate system of FIGS. 10A to 10C. The lower body member 38 may have any suitable cross-sectional shape or combination of shapes. In particular, when viewed in a cross-section that is normal to the X-axis, such as a cross-sectional plane that is parallel to the Y-Z plane of the reference coordinate system of FIGS. 10A to 10C, the lower body member 38 may have an arcuate, semi-circular, or partially-circular cross-sectional shape. That is, in cross section, the lower body member 38 may extend from the first body edge portion 46 to a second body edge portion 100 along a path or axis that may be a segment of a circle, such as one half of a circle, as illustrated in FIG. 10C. The cross-sectional shape may be further defined by a first edge 101 defined by the surface 51 (for example, the exterior surface) and second edge 102 defined by an interior surface 104. The cross-sectional shape may be bisected by a plane that contains the lower body axis 40 and that is parallel to the X-Z plane of the reference coordinate system of FIGS. 10A to 10C. The cross-sectional shape may be uniform or substantially uniform along the lower body axis 40 from the first end 42 of the lower body member 38 to the second end 44 of the lower body member 38.

As illustrated in FIG. 10A, the lower body member 38 may include the first lower body mating feature 48 that may be disposed along the first portion of the surface 51 of the lower body member 38. The first lower body mating feature 48 may be any feature that may engage the first upper lateral panel mating feature 34 (see FIG. 4A) such that the first upper lateral panel mating feature 34 is displaceable relative to the first lower body mating feature 48. For example, the first lower body mating feature 48 may be a first slot 90 disposed along the first portion of the surface 51 of the lower body member 38, and the first slot 90 of the lower body member 38 may extend along a first slot axis 106 from a first end 108 to a second end 110. The first slot axis 106 may extend parallel to the Y-axis of the reference coordinate system of FIGS. 10A to 10C when viewed along the Z-axis. However, the first slot axis 106 may conform in shape to the cross-sectional shape of the lower body member 38 when viewed in a cross-section that is normal to the X-axis, such as a cross-sectional plane that is parallel to the Y-Z plane of the reference coordinate system of FIGS. 10A to 10C. The first slot 90 may be defined by first and second lateral edges that are parallel, and the first and second ends 108, 110 may be defined by a first and second semi-circular edge, respectively. The first slot 90 may be disposed any distance from the first end 42 of the lower body member 38. In particular, the first slot axis 106 may be disposed a first distance D1 from the first end 42 of the lower body member 38.

Still referring to FIG. 10A, the lower body member 38 may include a second lower body mating feature 96 that may be disposed along a second portion of the surface 51 of the lower body member 38. The second lower body mating feature 96 may be any feature that may engage a second upper lateral panel mating feature 92 (see FIG. 4A) such that the second upper lateral panel mating feature 92 is displaceable relative to the second lower body mating feature 96. In some embodiments, the second lower body mating feature 96 may be identical to the first lower body mating feature 48. Specifically, the second lower body mating feature 96 may be the second slot 94 disposed along the second portion of the surface 51 of the lower body member 38, and the second slot 94 of the lower body member 38 may extend along a second slot axis 112 from a first end 114 to a second end 116. The second slot axis 112 may extend parallel to the Y-axis of the reference coordinate system of FIGS. 10A to 10C (and to the first slot axis 106) when viewed along the Z-axis. However, identical to the first slot axis 106, the second slot axis 112 may conform in shape to the cross-sectional shape of the lower body member 38 when viewed in a cross-section that is normal to the X-axis, such as a cross-sectional plane that is parallel to the Y-Z plane of the reference coordinate system of FIGS. 10A to 10C. The second slot axis 112 may be defined by first and second lateral edges that are parallel, and the first and second ends 114, 116 may be defined by a first and second semi-circular edge, respectively. The second slot axis 112 may be disposed any distance from the second end 44 of the lower body member 38. In particular, the second slot axis 112 may be disposed a second distance D2 from the second end 44 of the lower body member 38, and the second distance D2 may be equal to the first distance D1 between the first slot axis 106 and the first end 42 of the lower body member 38.

As illustrated in FIG. 1B, the first cannula subassembly 12 also includes the lower lateral panel 50 having a first panel edge 52 (see FIG. 1C) disposed at a first end of the lower lateral panel 50, and the first panel edge 52 (or any portion of the first end of the lower lateral panel 50) may be coupled to the lower body member 38 at or adjacent to the first body edge portion 46 of the lower body member 38. The first panel edge 52 of the lower lateral panel 50 may be coupled to the lower body member 38 in any suitable manner. For example, the first panel edge 52 of the lower lateral panel 50 may be fixedly coupled to the lower body member 38 by an adhesive, ultrasonic welding, or by mechanical coupling, such as snap-fit or friction fit coupling. In other embodiments, the first panel edge 52 of the lower lateral panel 50 may be coupled to the lower body member 38 by a hinge or flexible coupling. In still further embodiments, the first panel edge 52 of the lower lateral panel 50 may be integrally formed with the lower body member 38 at or adjacent to the first body edge portion 46 of the lower body member 38.

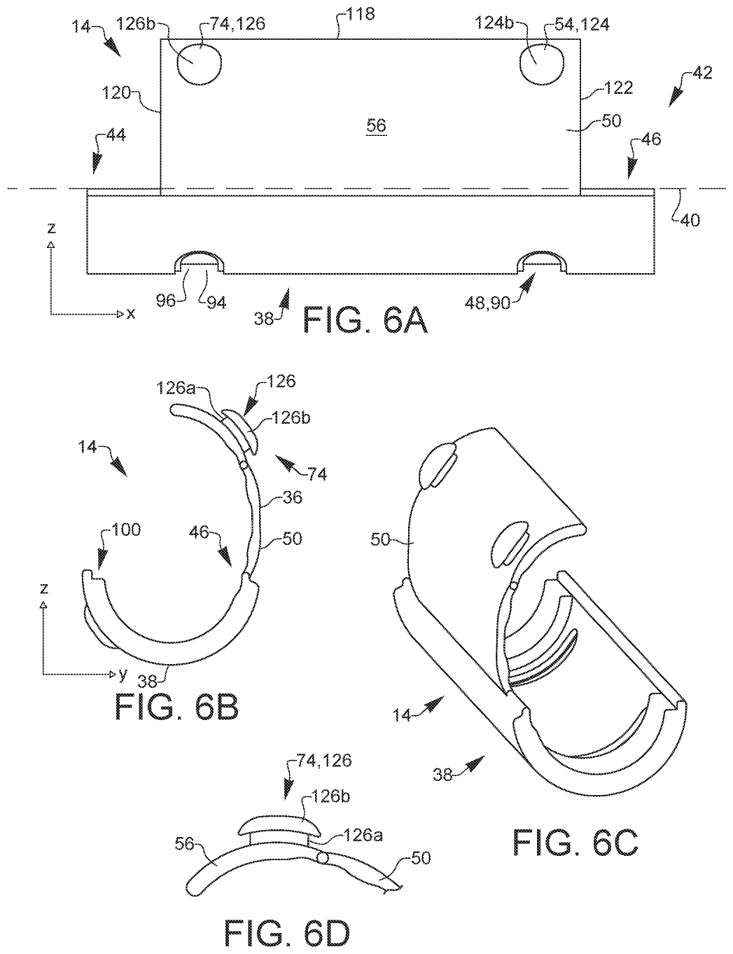

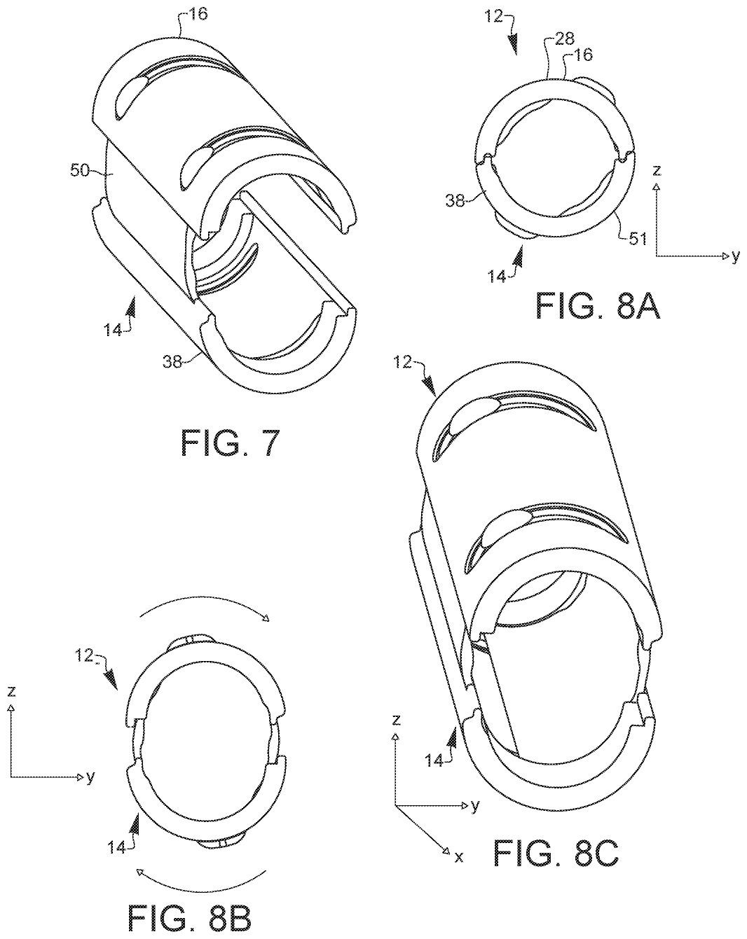

The lower lateral panel 50 may have any suitable shape or combination of shapes. In the embodiment of FIG. 6A, the lower lateral panel 50 may be defined by the first panel edge 52 and a second panel edge 118 that may be parallel (or substantially parallel) to the first panel edge 52 and/or the X-axis of the reference coordinate system of FIGS. 1E and 6A. The second panel edge 118 may define a second end of the lower lateral panel 50. The lower lateral panel 50 may be further defined by a first lateral edge 120 and a second lateral edge 122, where the first lateral edge 120 extends from a first end of the first panel edge 52 to a first end of the second panel edge 118 and the second lateral edge 122 extends from a second end of the first panel edge 52 to a second end of the second panel edge 118. Each of the first lateral edge 120 and the second lateral edge 122 may extend normal to the X-axis of the reference coordinate system of FIGS. 1E and 6A (i.e., along or parallel to the Z-axis). Accordingly, the lower lateral panel 50 may have a square or rectangular shape (or a substantially square or rectangular shape).

The lower lateral panel 50 may also have the first lower lateral panel mating feature 54 that is disposed on the first portion of the surface 56 (i.e., the outside surface) of the lower lateral panel 50. The first lower lateral panel mating feature 54 may be any feature that may engage the first upper body mating feature 26 of the upper body member 16 (illustrated in FIGS. 9A and 9B) such that the first lower lateral panel mating feature 54 is displaceable relative to the first upper body mating feature 26. In some embodiments, the first lower lateral panel mating feature 54 may be identical (or similar) to the first upper lateral panel mating feature 34 (illustrated in FIG. 4A). For example, the first lower lateral panel mating feature 54 may be a first protrusion 124 that may be adapted to be received in the first slot 64 disposed along the surface 28 of the upper body member 16 (illustrated in FIG. 1A). As illustrated in the cross-sectional view of FIG. 3C, the first protrusion 124 may have a base portion 124a that may be cylindrical and may have a diameter that is slightly smaller than the distance between the lateral edges of the first slot 64 such that the first protrusion 124 may translate longitudinally within the first slot 64. The first protrusion 124 may also have a top portion 124b coupled to the base portion 124a, and the top portion 124b may have the shape of a disk and may have a diameter that is larger than the distance between the lateral edges of the first slot 64 such that the first protrusion 124 is retained in the first slot 64 as the first protrusion 124 translates longitudinally within the first slot 64. The first protrusion 124 may be disposed adjacent to the second panel edge 118 (or the second end) of the lower lateral panel 50 and may be positioned to align with the first upper body mating feature 26 of the upper body member 16.

The lower lateral panel 50 may also have a second lower lateral panel mating feature 74 that is disposed on a second portion of the surface 56 (i.e., the outside surface) of the lower lateral panel 50. The second lower lateral panel mating feature 74 may be any feature that may engage the second upper body mating feature 72 of the upper body member 16 (illustrated in FIG. 1A) such that the second lower lateral panel mating feature 74 is displaceable relative to the second upper body mating feature 72. In some embodiments, the second lower lateral panel mating feature 74 may be identical to the first lower lateral panel mating feature 54. For example, the second lower lateral panel mating feature 74 may be a second protrusion 126 that may be adapted to be received in the second slot 76 disposed along the second portion of the surface 28 of the of the upper body member 16. As illustrated in FIG. 6D, the second protrusion 126 may be identical to the first protrusion 124 and may have a base portion 126a that may be cylindrical and may have a diameter that is slightly smaller than the distance between the lateral edges of the second slot 76 (illustrated in FIG. 9A) such that the second protrusion 126 may translate longitudinally within the second slot 76. The second protrusion 126 may also have a top portion 126b coupled to the base portion 126a, and the top portion 126b may have the shape of a disk and may have a diameter that is larger than the distance between the lateral edges of the second slot 76 (illustrated in FIG. 9A) such that the second protrusion 126 is retained in the second slot 76 as the second protrusion 126 translates longitudinally within the second slot 76. The second protrusion 126 may be disposed adjacent to the second panel edge 118 (or the second end) of the lower lateral panel 50 and may be positioned to align with the second upper body mating feature 72 of the upper body member 16.

The lower lateral panel 50 may be made from any suitable material, such as a flexible material. In some embodiments, the lower lateral panel 50 is configured to deform along an axis that is parallel to the lower body axis 40. In some embodiments, the lower lateral panel 50 may be made from a resilient material that may deform in a direction normal to the parallel to the lower body axis 40.

As previously explained, the first cannula subassembly 12 and the second cannula assembly 14 of the expandable cannula assembly 10 are displaceable between the first position (illustrated in FIGS. 1A to 2D) and the second position (illustrated in FIG. 8A). In the first position, the first protrusion 124 of the lower lateral panel 50 is disposed at or adjacent to the first end 68 of the first slot 64 of the upper body member 16, and the second protrusion 126 of the lower lateral panel 50 is disposed at or adjacent to the first end 80 of the second slot 76 of the upper body member 16. Also in the first position, the first protrusion 91 of the upper lateral panel 30 is disposed at or adjacent to the first end 108 of the first slot 90 of the lower body member 38, and the second protrusion 93 of the upper lateral panel 30 is disposed at or adjacent to the first end 114 of the second slot 94 of the lower body member 38. So configured, the first body edge portion 24 of the upper body member 16 is disposed offset (i.e., offset along the Z-axis of the reference coordinate system) from the second body edge portion 100 of the lower body member 38 and the second body edge portion 60 of the upper body member 16 is disposed offset (i.e., offset along the Z-axis of the reference coordinate system) from the first body edge portion 46 of the lower body member 38. In this first position (illustrated in FIG. 3A, or when viewed along the upper body axis 18, the lower body axis 40, or parallel to the X-axis of the reference coordinate system), the cross-sectional shape of the upper body member 16 and the upper lateral panel 30 (i.e., the first cross-sectional assembly shape of the first cannula subassembly 12) and the cross-sectional shape of the lower body member 38 and the lower lateral panel 50 (i.e., the first cross-sectional assembly shape of the second cannula subassembly 14) cooperate to form a first cross-sectional assembly shape that is an ovular cross-sectional shape or an oblong cross-sectional shape. The first cannula subassembly 12 and the second cannula subassembly 14 may also cooperate to have the shape of an oval or an oblong shape when viewed along the upper body axis 18, the lower body axis 40, or parallel to the X-axis of the reference coordinate system (such as the view of FIGS. 1C and 1F).

In the second position, the first protrusion 124 of the lower lateral panel 50 is disposed at or adjacent to the second end 70 of the first slot 64 of the upper body member 16, and the second protrusion 126 of the lower lateral panel 50 is disposed at or adjacent to the second end 82 of the second slot 76 of the upper body member 16. Also in the second position, the first protrusion 91 of the upper lateral panel 30 is disposed at or adjacent to the second end 110 of the first slot 90 of the lower body member 38, and the second protrusion 93 of the upper lateral panel 30 is disposed at or adjacent to the second end 116 of the second slot 94 of the lower body member 38. So configured, the first body edge portion 24 of the upper body member 16 is disposed at or adjacent to the second body edge portion 100 of the lower body member 38 and the second body edge portion 60 of the upper body member 16 is disposed at or adjacent to the first body edge portion 46 of the lower body member 38. In this second position (illustrated in FIG. 8A, or when viewed along the upper body axis 18, the lower body axis 40, or parallel to the X-axis of the reference coordinate system), the cross-sectional shape of the upper body member 16 (i.e., the second cross-sectional assembly shape of the first cannula subassembly 12) and the cross-sectional shape of the lower body member 38 (i.e., the second cross-sectional assembly shape of the second cannula subassembly 14) cooperate to form a second cross-sectional assembly shape that is a circular cross-sectional shape. The first cannula subassembly 12 and the second cannula subassembly 14 may also have the shape of a circle when viewed along the upper body axis 18, the lower body axis 40, or parallel to the X-axis of the reference coordinate system (such as the view of FIG. 8A).

In some embodiments, the first cannula subassembly 12 and the second cannula assembly 14 of the expandable cannula assembly 10 may be disposed in a third position (i.e., one or more intermediate positions, illustrated in FIG. 8B) between the first position and the second position. In the third position(s), the first protrusion 124 of the lower lateral panel 50 is disposed between the first end 68 and the second end of the first slot 64 of the upper body member 16, and the second protrusion 126 of the lower lateral panel 50 is disposed between the first end 80 and the second end 82 of the second slot 76 of the upper body member 16. Also in the third position, the first protrusion 91 of the upper lateral panel 30 is disposed between the first end 108 and the second end 110 of the first slot 90 of the lower body member 38, and the second protrusion 93 of the upper lateral panel 30 is disposed between the first end 114 and the second end 116 of the second slot 94 of the lower body member 38.

In some embodiments, one or more resilient members (not shown) may be coupled to one or both of the first cannula subassembly 12 and the second cannula assembly 14 of the expandable cannula assembly 10, and the one or more resilient members may act on one or both of the first cannula subassembly 12 and the second cannula assembly 14 to bias the expandable cannula assembly 10 (i.e., the first cannula subassembly 12 and the second cannula assembly 14) into the second position. In some embodiments, the one or more resilient members may be one or more elastic bands that surround the surface 28 of the upper body member 16 (see FIG. 8A) and the surface 51 of the lower body member 38 such that each of the one or more elastic bands surrounds the entire circumference of the upper body member 16 (see FIG. 8A) and the surface 51 of the lower body member 38 when the expandable cannula assembly 10 is in the second position. So positioned, the one or more elastic bands would apply a force to each of the upper body member 16 and the lower body member 38 that is substantially directed along or parallel to the Z-axis of the reference coordinate system, and this force would oppose the displacement of each of the upper body member 16 and the lower body member 38 from the second position into the first position.

In other embodiments, one or more springs or resilient members may be disposed between one or more portions of the upper body member 16 and the lower body member 38 to maintain or bias the upper body member 16 and the lower body member 38 (and the first cannula subassembly 12 and the second cannula assembly 14) into the second position.

In still other embodiments, all or a portion of the upper lateral panel 30 and the lower lateral panel 50 may be made from or comprise a resilient material that opposes the displacement of each of the upper body member 16 and the lower body member 38 from the second position into the first position. In such an embodiment, in the second position, the first protrusion 124 of the lower lateral panel 50 is disposed at or adjacent to the first end 68 of the first slot 64 of the upper body member 16, and the second protrusion 126 of the lower lateral panel 50 is disposed at or adjacent to the first end 80 of the second slot 76 of the upper body member 16. Also in the second position, the first protrusion 91 of the upper lateral panel 30 is disposed at or adjacent to the first end 108 of the first slot 90 of the lower body member 38, and the second protrusion 93 of the upper lateral panel 30 is disposed at or adjacent to the first end 114 of the second slot 94 of the lower body member 38. In such an embodiment, the position of the first and second protrusions 124, 126 of the lower lateral panel 50 and the first and protrusions 91, 93 of the upper lateral panel 30 do not change, but rather are retained in the first ends of the respective slots 64, 76, 90, 94 as the first cannula subassembly 12 and the second cannula assembly 14 are displaced from the second position into the first position. Thus, as the first cannula subassembly 12 and the second cannula assembly 14 are displaced from the second position into or towards the first (or third) position, the lower lateral panel 50 and the upper lateral panel 30 are stretched, and act to oppose the separating, or outward, displacement directed parallel to the Z-axis of the reference coordinate system. When the force causing the separating or outward displacement is removed, the lower lateral panel 50 and the upper lateral panel 30 act to return the first cannula subassembly 12 and the second cannula assembly 14 to or towards the second position.

When used during a procedure, the expandable cannula assembly 10 may be inserted into an incision of a patient in the second position to minimize trauma to the incision at the area of insertion. When a relatively large instrument 58 (illustrated in FIG. 11) is to be inserted through the expandable cannula assembly 10, a distal end of the instrument 58 is inserted into the proximal passage formed by the first end 20 of the upper body member 16 and the first end 42 of the lower body member 38. Although the first cannula subassembly 12 and the second cannula assembly 14 are illustrated in the first position prior to the insertion of the distal end of the instrument 58 in FIG. 11, contact between the distal end of the instrument 58 and the first end 20 of the upper body member 16 and the first end 42 of the lower body member 38 may cause the first cannula subassembly 12 and the second cannula assembly 14 to displace from the second position to the first position. While the expandable cannula assembly 10 is in the first position, the distal end of the instrument 58 may pass through the second end 22 of the upper body member 16 and the second end 44 of the lower body member 38 and be positioned at or adjacent to a desired treatment area.

When it is desired to remove the distal end of the instrument 58, the distal end of the instrument 58 may be displaced proximally until the distal end of the instrument 58 exits from the proximal passage formed by the first end 20 of the upper body member 16 and the first end 42 of the lower body member 38. In some embodiments (and particularly in embodiments with one or more resilient members acting one or both of the upper body member 16 and the lower body member 38), the lack of contact between the distal end of the instrument 58 and the first end 20 of the upper body member 16 and the first end 42 of the lower body member 38 may cause the first cannula subassembly 12 and the second cannula assembly 14 to displace from the first position to the second position. Accordingly, the time in which the expandable cannula assembly 10 is in the more traumatic first position is minimized, thereby correspondingly minimizing—or avoiding—trauma to the patient's incision.

In some embodiments, two or more expandable cannula assemblies 10 may be used and may be coupled such that the upper body axis 18a of the upper body member 16a of the first expandable cannula assembly 10a may be aligned with the upper body axis 18b of the upper body member 16b of the second expandable cannula assembly 10b, and so on. Accordingly, each segment of the overall assembly (i.e., the first expandable cannula assembly 10a out of an assembly of a first, second, and third expandable cannula assembly 10a, 10b, 10c) may expand or deploy from the second position to the first position as the distal end of the instrument 58 passes through that particular segment.

Various advantages of an expandable cannula assembly have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. An expandable cannula assembly comprising:
a first cannula subassembly comprising:
an upper body member that extends along an upper body axis from a first end to a second end, the upper body member having a first body edge portion that extends parallel to and offset from the upper body axis, wherein a first upper body mating feature is disposed along a first portion of a surface of the upper body member;
an upper lateral panel having a first panel edge that is coupled to the upper body member at or adjacent to the first body edge portion of the upper body member, and wherein a first upper lateral panel mating feature is disposed on a first portion of a surface of the upper lateral panel; and
a second cannula subassembly comprising:
a lower body member that extends along a lower body axis from a first end to a second end, the lower body member having a first body edge portion that extends parallel to and offset from the lower body axis, wherein a first lower body mating feature is disposed along a first portion of a surface of the lower body member; and
a lower lateral panel having a first panel edge that is coupled to the lower body member at or adjacent to the first body edge portion of the lower body member, and wherein a first lower lateral panel mating feature is disposed on a first portion of a surface of the lower lateral panel,
wherein the first lower lateral panel mating feature operatively engages the first upper body mating feature such that the first lower lateral panel mating feature is displaceable relative to the first upper body mating feature, and
the first upper lateral panel mating feature operatively engages the first lower body mating feature such that the first upper lateral panel mating feature is displaceable relative to the first lower body mating feature, and
wherein the first cannula subassembly and the second cannula assembly are displaceable between a first position and a second position, wherein in the first position, the first cannula subassembly and the second cannula assembly have a first cross-sectional assembly shape and in the second position, the first cannula subassembly and the second cannula assembly have a second cross-sectional assembly shape that is different than the first cross-sectional shape.

2. The expandable cannula assembly of claim 1, wherein:
the first upper body mating feature is a first slot is disposed along the first portion of the surface of the upper body member, the first slot of the upper body member extending along a first slot axis from a first end to a second end,
wherein the first upper lateral panel mating feature is a first protrusion disposed on the first portion of the surface of the upper lateral panel,
the first lower body mating feature is a first slot is disposed along the first portion of the surface of the lower body member, the first slot of the lower body member extending along a first slot axis from a first end to a second end, and
wherein the first upper lateral panel mating feature is a first protrusion disposed on the first portion of the surface of the upper lateral panel, and
wherein the first protrusion of the upper lateral panel is slidably disposed within the first slot of the lower body member, and wherein the first protrusion of the lower lateral panel is slidably disposed within the first slot of the upper body member.

3. The expandable cannula assembly of claim 2, wherein:
(a) when the first cannula subassembly and the second cannula assembly are in the first position, the first protrusion of the upper lateral panel is disposed at or adjacent to the first end of the first slot of the lower body member and the first protrusion of the lower lateral panel is disposed at or adjacent to the first end of the first slot of the upper body member, and
(b) when the first cannula subassembly and the second cannula assembly are in the second position, the first protrusion of the upper lateral panel is disposed at or adjacent to the second end of the first slot of the lower body member and the first protrusion of the lower lateral panel is disposed at or adjacent to the second end of the first slot of the upper body member.

4. The expandable cannula assembly of claim 1, the upper lateral panel further comprising a second panel edge, wherein each of the first panel edge and the second panel edge of the upper lateral panel extend parallel to and offset from the upper body axis, and
the lower lateral panel further comprising a second panel edge, wherein each of the first panel edge and the second panel edge of the lower lateral panel extend parallel to and offset from the lower body axis.

5. The expandable cannula assembly of claim 1, wherein the upper lateral panel is configured to deform along an axis that is parallel to the upper body axis, and the lower lateral panel is configured to deform along an axis that is parallel to the lower body axis.

6. The expandable cannula assembly of claim 1, wherein the upper body member includes a second body edge portion that extends parallel to and offset from the upper body axis, and wherein the lower body member includes a second body edge portion that extends parallel to and offset from the lower body axis,
wherein in the second position, the first body edge portion of the upper body member is disposed at or adjacent to the second body edge portion of the lower body member and the second body edge portion of the upper body member is disposed at or adjacent to the first body edge portion of the lower body member, and
wherein in the first position, the first body edge portion of the upper body member is disposed offset from the second body edge portion of the lower body member and the second body edge portion of the upper body member is disposed offset from the first body edge portion of the lower body member.

7. The expandable cannula assembly of claim 1, wherein when the first cannula subassembly and the second cannula assembly displace from the first position to the second position, the upper body member displaces in a direction normal to the upper body axis or the lower body member displaces in a direction normal to the lower body axis.

8. The expandable cannula assembly of claim 1, wherein the upper body member has a cross-sectional shape that is a segment of a first circle and the lower body member has a cross-sectional shape that is a segment of a second circle, and in the second position, the second cross-sectional assembly shape is a circular cross-sectional shape.

9. The expandable cannula assembly of claim 8, wherein in the first position, the cross-sectional shape of the upper body member and the upper lateral panel and the cross-sectional shape of the lower body member and the lower lateral panel cooperate to form a first cross-sectional assembly shape that is an ovular cross-sectional shape.

10. The expandable cannula assembly of claim 1, wherein:
a second upper body mating feature is disposed along a second portion of the surface of the upper body member,
a second upper lateral panel mating feature is disposed on a second portion of the surface of the upper lateral panel,
a second lower body mating feature is disposed along a second portion of the surface of the lower body member,
a second lower lateral panel mating feature is disposed on a second portion of the surface of the lower lateral panel,
the second lower lateral panel mating feature operatively engages the second upper body mating feature such that the second lower lateral panel mating feature is displaceable relative to the second upper body mating feature, and
the second upper lateral panel mating feature operatively engages the second lower body mating feature such that the second upper lateral panel mating feature is displaceable relative to the second lower body mating feature.

11. The expandable cannula assembly of claim 3, wherein:
a second upper body mating feature is disposed along a second portion of the surface of the upper body member,
a second upper lateral panel mating feature is disposed on a second portion of the surface of the upper lateral panel,
a second lower body mating feature is disposed along a second portion of the surface of the lower body member,
a second lower lateral panel mating feature is disposed on a second portion of the surface of the lower lateral panel,
the second lower lateral panel mating feature operatively engages the second upper body mating feature such that the second lower lateral panel mating feature is displaceable relative to the second upper body mating feature, and
the second upper lateral panel mating feature operatively engages the second lower body mating feature such that the second upper lateral panel mating feature is displaceable relative to the second lower body mating feature.

12. The expandable cannula assembly of claim 11, wherein:
the second upper body mating feature is a second slot is disposed along the second portion of the surface of the upper body member, the second slot of the upper body member extending along a second slot axis from a first end to a second end,
wherein the second upper lateral panel mating feature is a second protrusion disposed on the second portion of the surface of the upper lateral panel,
the second lower body mating feature is a second slot is disposed along the second portion of the surface of the lower body member, the second slot of the lower body member extending along a second slot axis from a first end to a second end, and
wherein the second upper lateral panel mating feature is a second protrusion disposed on the second portion of the surface of the upper lateral panel, and
wherein the second protrusion of the upper lateral panel is slidably disposed within the second slot of the lower body member, and wherein the second protrusion of the lower lateral panel is slidably disposed within the second slot of the upper body member, and
(a) when the first cannula subassembly and the second cannula assembly are in the second position, the second protrusion of the upper lateral panel is disposed at or adjacent to the second end of the second slot of the lower body member and the second protrusion of the lower lateral panel is disposed at or adjacent to the second end of the second slot of the upper body member, and
(b) when the first cannula subassembly and the second cannula assembly are in the first position, the second protrusion of the upper lateral panel is disposed at or adjacent to the first end of the second slot of the lower body member and the second protrusion of the lower lateral panel is disposed at or adjacent to the first end of the second slot of the upper body member.

13. An expandable cannula assembly comprising:
a first cannula subassembly comprising:
an upper body member that extends along an upper body axis from a first end to a second end, the upper body member having a first body edge portion that extends parallel to and offset from the upper body axis, wherein a first slot is disposed along a first portion of a surface of the upper body member, the first slot of the upper body member extending along a first slot axis from a first end to a second end;
an upper lateral panel having a first panel edge that coupled to the upper body member at or adjacent to the first body edge portion of the upper body member, and wherein a first protrusion is disposed on first portion of a surface of the upper lateral panel; and
a second cannula subassembly comprising:
a lower body member that extends along a lower body axis from a first end to a second end, the lower body member having a first body edge portion that extends parallel to and offset from the lower body axis, wherein a first slot is disposed along a first portion of a surface of the lower body member, the first slot of the lower body member extending along a first slot axis from a first end to a second end; and
a lower lateral panel having a first panel edge that is coupled to the lower body member at or adjacent to the first body edge portion of the lower body member, and wherein a first protrusion is disposed on a first portion of a surface of the lower lateral panel,
wherein the first protrusion of the upper lateral panel is slidably disposed within the first slot of the lower body member, and wherein the first protrusion of the lower lateral panel is slidably disposed within the first slot of the upper body member, and
wherein the first cannula subassembly and the second cannula assembly are displaceable between a first position and a second position, wherein in the first position, the first cannula subassembly and the second cannula assembly have a first cross-sectional assembly shape and in the second position, the first cannula subassembly and the second cannula assembly have a second cross-sectional assembly shape that is different than the first cross-sectional shape.

14. The expandable cannula assembly of claim 13, wherein:
(a) when the first cannula subassembly and the second cannula assembly are in the first position, the first protrusion of the upper lateral panel is disposed at or adjacent to the first end of the first slot of the lower body member and the first protrusion of the lower lateral panel is disposed at or adjacent to the first end of the first slot of the upper body member, and
(b) when the first cannula subassembly and the second cannula assembly are in the second position, the first protrusion of the upper lateral panel is disposed at or adjacent to the second end of the first slot of the lower body member and the first protrusion of the lower lateral panel is disposed at or adjacent to the second end of the first slot of the upper body member.

15. The expandable cannula assembly of claim 14, wherein:
a second slot is disposed along a second portion of the surface of the upper body member, the second slot of the upper body member extending along a second slot axis from a first end to a second end,
a second protrusion is disposed on second portion of the surface of the upper lateral panel,
a second slot is disposed along a second portion of the surface of the lower body member, the second slot of the lower body member extending along a second slot axis from a first end to a second end, and
a second protrusion is disposed on a second portion of a surface of the lower lateral panel, and wherein:
(a) when the first cannula subassembly and the second cannula assembly are in the second position, the second protrusion of the upper lateral panel is disposed at or adjacent to the second end of the second slot of the lower body member and the second protrusion of the lower lateral panel is disposed at or adjacent to the second end of the second slot of the upper body member, and
(b) when the first cannula subassembly and the second cannula assembly are in the first position, the second protrusion of the upper lateral panel is disposed at or adjacent to the first end of the second slot of the lower body member and the second protrusion of the lower lateral panel is disposed at or adjacent to the first end of the second slot of the upper body member.
